(19) United States
(12) Patent Application Publication
(10) Pub. No.: US 2012/0314840 A1
(43) Pub. Date: Dec. 13, 2012

(54) RADIATION THERAPY DEVICE AND METHOD FOR GENERATING A RESOLUTION ENHANCEMENT IN IRRADIATED RADIATION-EXPOSURE FIELDS

(76) Inventor: Oliver Heid, Erlangen (DE)

(21) Appl. No.: 13/579,914

(22) PCT Filed: Feb. 2, 2011
(86) PCT No.: PCT/EP2011/051460

§ 371 Date: Aug. 17, 2012

(30) Foreign Application Priority Data
Feb. 24, 2010 (DE) 10 2010 009 018.2

(51) Int. Cl. A61N 5/10

(52) U.S. Cl. 378/65

(57) ABSTRACT

A radiation therapy device may include: a radiation source that directs a beam to a target volume from at least two opposite directions, a collimator having multiple elements for localizing the treatment beam to generate a radiation-exposure field, wherein an expansion of the collimator elements predetermines a resolution of the radiation-exposure field, and an offset unit affects the irradiation of opposing radiation-exposure fields at an offset such that the two opposing radiation-exposure fields are offset from each other by a fraction of the resolution. Furthermore, a method for generating a resolution enhancement in irradiated radiation-exposure fields may include: generating a first radiation-exposure field using a collimator which delimits a beam emitted from a first spatial direction; and generating a second radiation-exposure field using the collimator which delimits a further beam emitted from a second spatial direction, the second radiation-exposure field being offset from the first radiation-exposure field by a fraction of the resolution.

### CROSS-REFERENCE TO RELATED APPLICATIONS

This application is a U.S. National Stage Application of International Application No. PCT/EP2011/051460 filed Feb. 2, 2011, which designates the United States of America, and claims priority to DE Patent Application No. 10 2010 009 018.2 filed Feb. 24, 2010. The contents of which are hereby incorporated by reference in their entirety.

### TECHNICAL FIELD

This disclosure relates to a radiation therapy device and a method for generating an enhanced resolution in irradiated radiation-exposure fields and, associated therewith, the irradiated dose distribution.

### BACKGROUND

Radiation therapy devices are used in a known manner to treat diseases such as tumors for instance. High energy x-rays are herewith usually irradiated onto a target volume to be irradiated, such as for instance a human body or a phantom for research or maintenance purposes. The dose distribution is adjusted to the target volume to be irradiated.

This is usually achieved by high-resolution collimators, which delimit the beam laterally. The higher resolution a collimator, i.e. the finer a radiation-exposure field generated by the collimator can be graded, the more accurately a desired dose distribution can be applied.

High resolution collimators nevertheless require a complicated construction and are comparatively expensive.

### SUMMARY

In one embodiment, a radiation therapy device comprises: a radiation source, from which for radiation-exposure purposes a beam can be directed onto a target volume from at least two opposite directions; a collimator having a plurality of collimator elements for localizing the treatment beam in order to generate a radiation-exposure field, wherein the expansion of the collimator elements predetermines a resolution of the radiation-exposure field; and an offset unit, which causes the irradiation of opposing radiation-exposure fields to be effected at an offset such that the two opposing irradiated radiation-exposure fields are offset relative to each other by a fraction of the resolution.

In a further embodiment, the offset unit is embodied to effect an offset of a quarter or a half of the resolution predetermined by the collimator elements. In a further embodiment, the radiation source and the collimator are rotatably mounted about an axis of rotation, wherein the expansion of the collimator elements predetermines a resolution of the radiation-exposure field in the direction of the axis of rotation and wherein the offset unit is embodied to effect an offset along the axis of rotation. In a further embodiment, the rotation of the radiation source and the collimator takes place helically. In a further embodiment, the radiation source and the collimator are rotatably mounted about an axis of rotation, wherein the expansion of the collimator elements predetermines a resolution of the radiation-exposure field in a direction at right angles to the axis of rotation, and wherein the offset unit is embodied to effect an offset at right angles to the axis of rotation. In a further embodiment, the offset unit causes the radiation-exposure field applied by the collimator to be arranged relative to an isocentrically aligned radius such that the radiation-exposure field is offset by a quarter of the resolution of the radiation-exposure field relative to the radius.

In another embodiment, a method for generating a resolution enhancement in irradiated radiation-exposure fields in a radiation therapy device, comprises: generating a first radiation-exposure field with the aid of a radiation source and a collimator, which delimits a beam of the radiation source to be applied and emitted from a first spatial direction and which includes a plurality of collimator elements, which predetermine a resolution of the radiation-exposure field, and generating a second radiation-exposure field with the aid of the collimator, which delimits a further beam emitted from a second spatial direction, wherein the second radiation-exposure field is offset by a fraction of the resolution relative to the first radiation-exposure field.

In a further embodiment, the second radiation-exposure field is offset by a quarter or a half of the resolution relative to the first radiation-exposure field. In a further embodiment, the radiation source and the collimator are rotatably mounted about an axis of rotation, wherein the expansion of the collimator elements predetermines a resolution of the radiation-exposure field in the direction of the axis of rotation, and wherein the radiation-exposure fields are offset relative to one another along the axis of rotation, wherein the rotation of the radiation source and of the collimator takes place in particular helically about the axis of rotation. In a further embodiment, the radiation source and the collimator are rotatably mounted about an axis of rotation, wherein the expansion of the collimator elements predetermines a resolution of the radiation-exposure field in a direction at right angles to the axis of rotation, and the offset takes place at right angles to the axis of rotation. In a further embodiment, the radiation-exposure field applied by the collimator is arranged relative to an isocentrically aligned radius such that the radiation-exposure field is offset by a quarter of the resolution of the radiation-exposure field relative to the radius.

### BRIEF DESCRIPTION OF THE DRAWINGS

Example embodiments will be explained in more detail below with reference to figures, in which:

FIG. 1 shows a representation to explain the principle of how a resolution enhancement can be achieved,

FIG. 2 shows a representation of how the resolution enhancement can be achieved in a radiation therapy device,

FIG. 3 shows a representation of how the resolution enhancement can be achieved in a radiation therapy device according to another embodiment, and

FIG. 4 shows the offset of the radiation-exposure field in the embodiment shown in FIG. 3 with respect to a central, isocentric radius.

### DETAILED DESCRIPTION

Some embodiments provide a radiation therapy device that allows for a high resolution of the irradiated dose distribution even with a simpler construction of a collimator. Furthermore, some embodiments provide a corresponding method for generating a resolution enhancement in irradiated radiation-exposed fields.

In one embodiment, a radiation therapy device includes:
a radiation source, for instance an x-ray radiation source, from which for radiation exposure purposes a beam can be directed onto a target volume from at least two opposite directions,
a collimator having a plurality of collimator elements for localizing the treatment beam in order to generate a radiation-exposure field, wherein the expansion of the collimator elements predetermines a resolution of the radiation-exposure field, and
an offset unit, which causes the irradiation of opposing radiation-exposure fields to be effected at an offset such that the two opposing radiation-exposure fields are offset relative to each other by a fraction of the resolution.

The dose distribution in the irradiation volume may be composed by sequentially using two beam bundles from opposite spatial directions, wherein the axes of the beam bundles are offset by a fraction, e.g. by half or a quarter, of the resolution of the collimator. The resolution of the dose distribution which can be achieved in the irradiation volume is effectively doubled as a result. This may provide an improved local dose distribution in radiation therapy devices, such as x-ray radiation therapy devices.

An advantage of a radiation therapy device of this type is a halving and/or quartering of the collimator elements needed to achieve a specific resolution of the dose distribution, e.g. disks or needles, depending on the structure of the collimator. Significant simplification can therefore be achieved, reducing the complexity of the beam forming mechanism.

The irradiation from opposite directions can take place sequentially.

With a collimator, for instance by expanding the collimator elements, for instance the width of a disk, a minimal resolution can be predetermined in a direction of a two-dimensional radiation-exposure field or depending on the structure of the collimator also in the two directions of the two-dimensional radiation-exposure field. This resolution can be increased if, with the second radiation-exposure field which is irradiated from the opposite direction, an offset of the beam bundle by a fraction of the resolution of the collimator takes place in precisely this direction.

The radiation therapy device may comprise a cylinder geometry, i.e., such that the radiation source and the collimator are rotatably mounted about an axis of rotation about an isocenter. The collimator can be arranged such that the expansion of the collimator elements predetermines a minimal resolution of the radiation-exposure field in the direction of the axis of rotation. The offset unit can then be embodied to effect an offset along the axis of rotation. Axial offsetting takes place here by offsetting in the direction of rotation, e.g. by a quarter or a half of the resolution.

This can be effected for instance by the rotation of the radiation source and of the collimator taking place in a helical manner about the axis of rotation. With a half revolution, an offset of the radiation-exposure field can take place for instance by a half or a quarter of the resolution of the radiation-exposure field. This can be achieved for instance by the collimator and the beam source simultaneously executing a linear movement along the axis of rotation during rotation. The movement is therefore helical.

The radiation source and the collimator can however also be rotatably mounted about an axis of rotation about an isocenter, and the collimator can be arranged in this case such that the expansion of the collimator elements predetermines a minimal resolution of the radiation-exposure field in a direction at right angles to the axis of rotation. The offset unit is then embodied to effect an offset at right angles to the axis of rotation.

This can be realized for instance by the offset unit causing the field applied by the collimator to be arranged relative to an isocentrically aligned radius such that the field is offset by a quarter of the resolution of the radiation-exposure field relative to the radius. By the field being offset by a quarter, an offset by a total of half of the resolution of the radiation-exposure field is produced during irradiation from opposite directions. This embodiment may be advantageous in that the offset with oppositely irradiated fields is already effected purely by means of the geometric arrangement of the collimator.

In one embodiment, a method for generating a resolution enhancement in irradiated radiation-exposure field in a radiation therapy device may include the following steps:
generating a first radiation-exposure field with the aid of a collimator, which delimits a beam to be applied and emitted from a first spatial direction and includes a plurality of collimator elements, which predetermine a resolution of the radiation-exposure field, and
generating a second radiation-exposure field with the aid of the collimator, which delimits a further beam emitted from a second spatial direction,
   wherein the second radiation-exposure field is offset by a fraction of the resolution relative to the first radiation-exposure field.

The second radiation-exposure field can be irradiated sequentially relative to the first radiation-exposure field and offset by a quarter or a half of the resolution. The radiation source and the collimator can be rotatably mounted about an axis of rotation about an isocenter, and the radiation-exposure fields can be offset relative to one another along the axis of rotation.

The rotation of the radiation source and the collimator can take place helically about the axis of rotation.

The radiation source and the collimator can be rotatably mounted about an axis of rotation, wherein by expanding the collimator elements, a resolution of the radiation-exposure field is predetermined in a direction at right angles to the axis of rotation and the offset takes place at right angles to the axis of rotation. E.g. the field applied by the collimator can be arranged relative to an isocentrically aligned radius such that the field is offset by a quarter of the resolution of the radiation-exposure field relative to the radius.

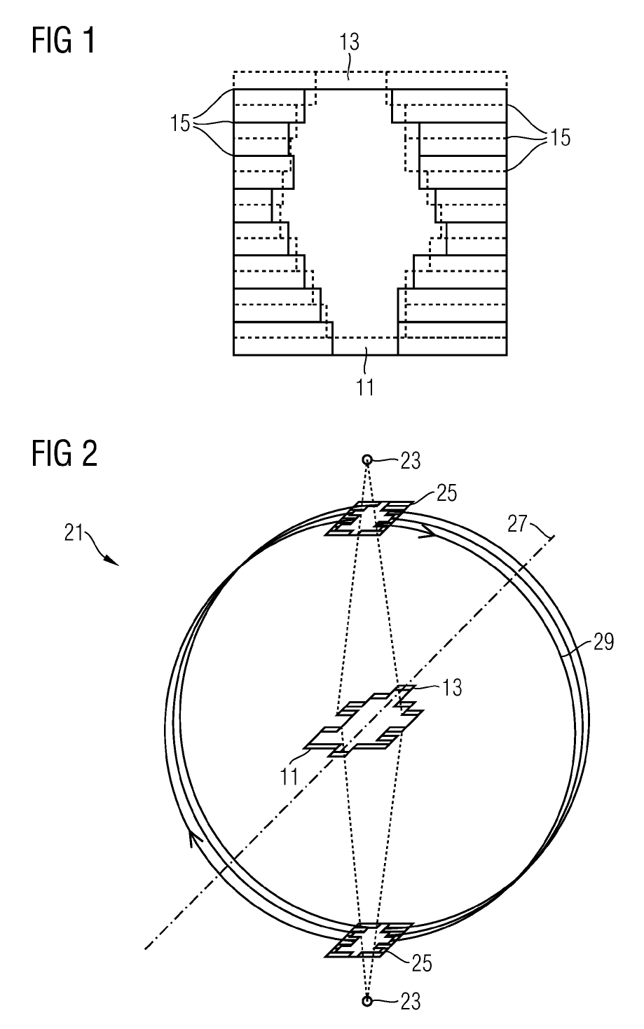

FIG. 1 shows a first radiation-exposure field 11 and an overlaid second radiation-exposure field 13. The first radiation-exposure field 11 is shown by a continuous line, the second radiation-exposure field 13 is shown by a dashed line.

Similarly drawn are the projections of the collimator elements 15, which each generate the radiation-exposure field.

The first radiation-exposure field 11 is applied in the irradiation volume, by a beam being applied from a first direction and being delimited by a collimator. The width of the collimator elements, e.g. disks, restricts the resolution of the first radiation-exposure field 11 in one direction.

The irradiation of the second radiation-exposure field 13 takes place from an opposite spatial direction namely such that the second radiation-exposure field 13, in the direction in which the resolution restriction is predetermined by the collimator structure, is offset. An offset by half of the resolution is shown here, and other fractions are also possible as the offset.

The dose distribution which is composed of the two radiation-exposure fields 11, 13, has a resolution as a result which is twice as large as the resolution predetermined by the collimator.

FIG. 2 shows a radiation therapy device 21, in which a radiation source 23 and a collimator 25 can be rotated about an axis of rotation 27. Further components of the radiation therapy device 21 are not shown for the sake of simplicity.

The rotation of the radiation source 23 and of the collimator 25 about the target volume (not shown) to be irradiated takes place along a helical path 29. The path 29 is selected such that when irradiating the radiation-exposure fields 11, 13 from different directions, an offset of the radiation-exposure fields 11, 13 takes place precisely by half of the resolution predetermined by the collimator 25. In this case, the offset unit, which provides for the offset of the radiation-exposure fields, corresponds to the mechanism which enables the helical path movement of the radiation source 23 and of the collimator 25.

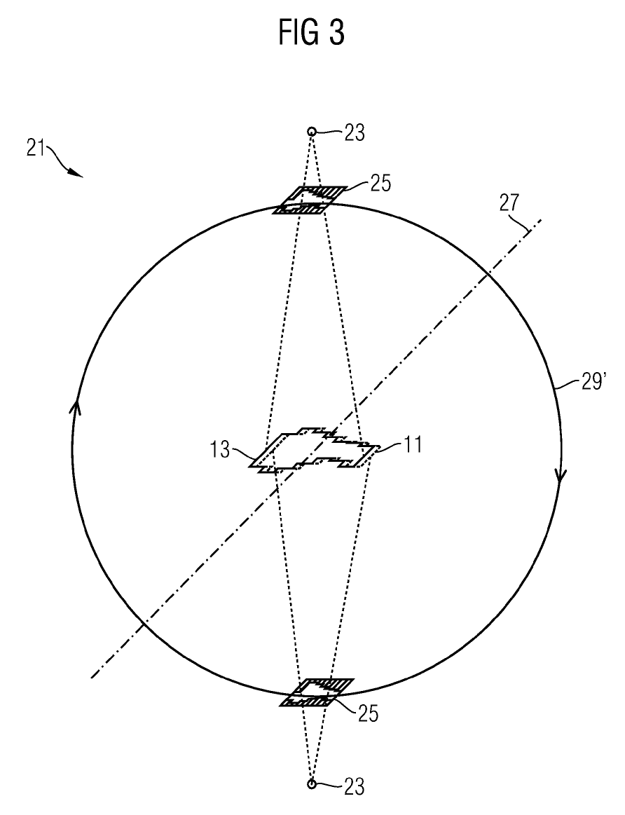

FIG. 3 shows a radiation therapy device 21, in which a radiation source 23 and a collimator 25 can be rotated about an axis of rotation 27. Further components of the radiation therapy device 21 are not shown for the sake of simplicity.

The rotation of the radiation source 23 and of the collimator 25 about the target volume to be irradiated (not shown) takes place along a circular path 29′. The irradiation of the radiation-exposure fields 11, 13 is selected such that when irradiating the radiation-exposure fields 11, 13 from different directions, an offset of the radiation-exposure fields 11, 13 takes place precisely by half of the resolution predetermined by the collimator 25. The offset takes place along a direction which is at right angles to the axis of rotation 27.

In this case, the offset unit which provides for the offset of the radiation-exposure fields, corresponds to the mechanism which enables the irradiation of a field offset by a quarter of the resolution relative to an isocentric, imaginary radius 31.

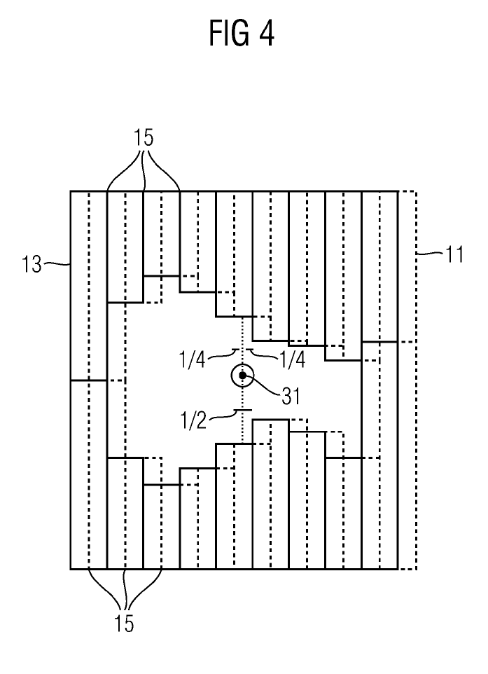

This is explained again with the aid of FIG. 4. As a result of a radiation-exposure field being irradiated offset by a quarter relative to an isocentric radius, with an opposite irradiation an offset of in total half of the resolution of the radiation-exposure field is achieved.

The embodiments according to FIG. 3 and FIG. 4 can be combined with one another, for instance with a collimator which is created such that a resolution limit is predetermined in both directions of its radiation-exposure field.

### LIST OF REFERENCE CHARACTERS

11 first radiation-exposure field
13 second radiation-exposure field
15 projections of the collimator elements
21 radiation therapy device
23 radiation source
25 collimator
27 axis of rotation
29 helical path
29′ circular path
31 isocentric radius

What is claimed is:

1. A radiation therapy device comprising:
a radiation source configured to emit a beam toward a target volume from at least two opposite directions,
a collimator having a plurality of collimator elements for localizing the beam to generate a radiation-exposure field, wherein an expansion of the collimator elements predetermines a resolution of the radiation-exposure field,
wherein the radiation therapy device is configured such that during operation a dose distribution in an irradiation volume is composed by sequentially using two beam bundles from opposing spatial directions, and
an offset unit configured to affect the irradiation of opposing radiation-exposure fields at an offset such that the two opposing irradiated radiation-exposure fields are offset relative to each other by a fraction of the resolution, wherein the axes of the two beam bundles are offset by a fraction of the resolution relative to each other.

2. The radiation therapy device of claim 1, wherein the offset unit is configured to effect an offset of a quarter or a half of the resolution predetermined by the collimator elements.

3. The radiation therapy device of claim 1, wherein:
the radiation source and the collimator are rotatably mounted about an axis of rotation, and
the expansion of the collimator elements predetermines a resolution of the radiation-exposure field in the direction of the axis of rotation and wherein the offset unit is embodied to effect an offset along the axis of rotation.

4. The radiation therapy device of claim 3, wherein the rotation of the radiation source and the collimator is helical.

5. The radiation therapy device of claim 1, wherein:
the radiation source and the collimator are rotatably mounted about an axis of rotation,
the expansion of the collimator elements predetermines a resolution of the radiation-exposure field in a direction at right angles to the axis of rotation, and
the offset unit is configured to effect an offset at right angles to the axis of rotation.

6. The radiation therapy device of claim 5, wherein the offset unit causes the radiation-exposure field applied by the collimator to be arranged relative to an isocentrically aligned radius such that the radiation-exposure field is offset by a quarter of the resolution of the radiation-exposure field relative to the radius.

7. A method for generating a resolution enhancement in irradiated radiation-exposure fields in a radiation therapy device, comprising:
generating a first radiation-exposure field using a radiation source and a collimator, which delimits a beam bundle of the radiation source to be applied and emitted from a first spatial direction and which includes a plurality of collimator elements, which predetermine a resolution of the radiation-exposure field, and
generating a second radiation-exposure field using the collimator, which delimits a further beam bundle emitted from a second spatial direction,
wherein the second radiation-exposure field is offset by a fraction of the resolution relative to the first radiation-exposure field, and wherein the axes of the two beam bundles are offset by a fraction of the resolution relative to each other.

8. The method of claim 7, wherein the second radiation-exposure field is offset by a quarter or a half of the resolution relative to the first radiation-exposure field.

9. The method of claim 7, wherein:
the radiation source and the collimator are rotatably mounted about an axis of rotation,
the expansion of the collimator elements predetermines a resolution of the radiation-exposure field in the direction of the axis of rotation, and
the radiation-exposure fields are offset relative to one another along the axis of rotation, wherein the rotation of the radiation source and of the collimator occurs helically about the axis of rotation.

10. The method of claim 7, wherein:
the radiation source and the collimator are rotatably mounted about an axis of rotation,
the expansion of the collimator elements predetermines a resolution of the radiation-exposure field in a direction at right angles to the axis of rotation, and
the offset occurs at right angles to the axis of rotation.

11. The method of claim 7, wherein the radiation-exposure field applied by the collimator is arranged relative to an isocentrically aligned radius such that the radiation-exposure field is offset by a quarter of the resolution of the radiation-exposure field relative to the radius.
